(19) United States
(12) Patent Application Publication
(10) Pub. No.: US 2025/0170198 A1
(43) Pub. Date: May 29, 2025

(54) LOTUS LEAF-DERIVED EXOSOMES HAVING DISPERSION STABILITY AND USE THEREOF IN ALLEVIATING INFLAMMATORY RESPONSE OR IN WOUND HEALING

(71) Applicants: Ching Yao KUO, New Taipei City (TW); BO HUI BIOTECH CO., LTD., New Taipei City (TW)

(72) Inventor: Ching Yao KUO, New Taipei City (TW)

(21) Appl. No.: 18/957,509

(22) Filed: Nov. 22, 2024

(30) Foreign Application Priority Data
Nov. 23, 2023 (TW) 112145401

(51) Int. Cl. A61K 36/48; A61K 9/50; A61P 29/00

(52) U.S. Cl. CPC A61K 36/48; A61K 9/5068; A61P 29/00; A61K 2236/53

(57) ABSTRACT

The present invention provides a lotus-derived extracellular vesicle (LDEV), which are extracted from lotus leaves by a group selected from the following extraction methods: polymer precipitation method, ultracentrifugation method, ultrafiltration method, density gradient centrifugation method and size-exclusion chromatography. The present invention also provides an anti-inflammatory composition containing the LDEV and the use of the LDEV. The LDEV extracted through different separation methods in the present invention have similar particle sizes and stable zeta potentials. In addition, the present invention has experimentally confirmed that the LDEV can be used to alleviate inflammatory reactions or wound healing, and can further be prepare as anti-inflammatory or wound healing drugs, compositions or nutritional supplements.

### BACKGROUND OF THE INVENTION

### 1. Technical Field

The present invention relates to plant-derived exosomes and more particularly to lotus leaf-derived exosomes.

### 2. Description of Related Art

Extracellular vesicles (EVs) are vesicles produced by cells and having a lipid bilayer structure. Existing widely in living organisms, including animals, plants, and microorganisms, EVs were originally regarded as cell fragments responsible for taking waste out of cells. However, as these vesicles carry a large amount of proteins, lipids, RNAs, and DNAs, are of sizes ranging from 50 to 1000 nm, and can pass through cell membranes freely, they are today no longer viewed only as a carrier of intracellular waste, but also as an important part of intercellular communication.

Based on their forming methods, EVs can be divided into two major types: exosomes and microvesicles. Exosomes range in size from 30 to 150 nm and are essentially intraluminal vesicles (ILVs) in multi-vesicular bodies (MVBs). The ILVs are released from cells as exosomes when the MVBs fuse with the cell membranes. Microvesicles, on the other hand, range in size from 50 to 1000 nm and are released from cells through a budding process. With the increase of studies on EVs in the past decade, scientists have gradually found that EVs have a variety of bioactivity and participate in various physiological and pathological processes in living organisms, such as blood coagulation, angiogenesis, immunoregulation, and inflammation. Studies have also shown that different types of cells will regulate the biosynthesis methods of EVs according to their respective physiological states in order to release specific lipids, proteins, and nucleic acids, and that in consequence, EVs can be used as biomarkers for treating cancer. In addition, thanks to their feature of being able to pass through cells, EVs can be used to treat diseases by serving as nanovehicles for drugs.

Plant-derived exosome-like nanoparticles (PDENs) are nanoparticles that can be found in plants, either in the cytoplasm or in the extracellular matrix, and that are similar to exosomes. Recently, with the advancement of research, more and more studies have proved that PDENs have various bioactive functions such as anti-oxidation, anti-inflammation, enteric bacterial flora regulation, and the prevention/treatment of cancer. PDENs can also be used as drug vehicles. Many researchers have successfully extracted PDENs from, for example, ginger, grape, grapefruit, lemon, apple, cherry, strawberry, tomato, wheat, carrot, Chinese cabbage, and cabbage.

While PDENs have huge application potential in clinical medicine, it is a great challenge to extract them from living organisms in an efficient and precise manner because, like other exosomes, PDENs are nanoscale, and mostly heterogeneous, particles. Currently, due to a lack of standard purification method, the purification of PDENs is carried out with reference to the purification methods of exosomes in mammalian cells. However, as the exosome purification method changes, so will the membrane protein level, and contents, of the exosomes obtained. In addition, studies have shown that the lipid compositions of PDENs are a key factor in determining the target cells of exosomes. For example, exosome-like nanoparticles derived from grape can only be delivered to cells of the intestinal tract, but lipid-modified grape-derived exosome-like nanoparticles can be delivered to brain cells through the nasal cavity. All the foregoing studies indicate that the establishment of a standard purification method capable of stabilizing the quality of PDENs is an issue in clinical applications that demands solution.

Lotus (Nelumbo nucifera Gaertn.), also known as Indian lotus, is a perennial aquatic plant belonging to the genus Nelumbo in the family Nelumbonaceae and widely planted in East Asia and India. It is a decorative plant and can also be used to prepare food and drinks. In traditional Chinese medicine, lotus has had a long history of use in treating diseases such as vomiting blood, bleeding from the nose, and high levels of blood lipids. Studies have also shown that lotus has a variety of pharmacological and physiological activity, e.g., in liver protection, anti-oxidation, the treatment of diarrhea, the treatment of viral infections, immunoregulation, and body fat reduction.

The entire lotus plant, including the leaf, flower, rhizome, and seed pod, is useful, which makes lotus a crop of high economic value. The pharmacological functions of lotus stem from the various bioactive substances that are richly contained in lotus, and these substances include polysaccharides, essential oils, flavonoids, alkaloids, and triterpenoids, in which flavonoids and alkaloids have been studied the most. In particular, the separation, purification, and activity analysis of the flavonoid compounds in lotus leaves have been major objectives of research on lotus leaves.

### BRIEF SUMMARY OF THE INVENTION

The contents of this section aim to provide a simplified version of the contents of the present invention so that a reader will have a basic understanding of the invention. The brief summary of the invention is not a complete description of the invention and is not intended to point out the important/key elements of an embodiment of the invention or to define the scope of the invention.

Recent studies on PDENs have shed more and more light on the functions and applications of PDENs. Although several kinds of fruits and vegetables have been reported, there has been no research on lotus leaf-derived exosome-like nanoparticles. In view of the multiple bioactive substances in lotus leaves, it is worthwhile to evaluate whether or not lotus leaves contain PDENs that have bioactivity. PDENs are known to be able to carry the mRNAs, miRNAs, bioactive substances, and proteins of various plants into, and thereby improve the cell viability of, animal cells. Previous studies have also shown that lotus leaves contain different active substances and can be used as health food, or nutritional supplements. Therefore, the inventor of the present invention conducted a series of experiments to find out whether lotus leaves also contain exosome-like nanoparticles or not, to evaluate the effects of different plant-derived EV extraction methods on lotus leaf-derived exosome particles, to establish a purification method and qualitative standard for lotus leaf-derived exosomes, and to analyze whether or not lotus leaf-derived exosome nanoparticles contain effective bioactive substances.

In one embodiment of the present invention, lotus leaf-derived exosomes, extracted from lotus leaves by an extraction method selected from the group consisting of: polymer precipitation, ultracentrifugation, ultrafiltration, density-gradient centrifugation, and size-exclusion chromatography.

In one embodiment of the present invention, the extraction method is the ultrafiltration.

In one embodiment of the present invention, the lotus leaf-derived exosomes have particle sizes in a range from 50 to 300 nm.

In one embodiment of the present invention, the lotus leaf-derived exosomes have a zeta potential lower than −20 mV.

In one embodiment of the present invention, the zeta potential of the lotus leaf-derived exosomes is lower than −30 mV.

In one embodiment of the present invention, the lotus leaf-derived exosomes are used to alleviate inflammatory response or heal a wound.

In one embodiment of the present invention, the inflammatory response is caused by a lipopolysaccharide (LPS).

In another aspect, the present invention also provides an anti-inflammatory composition, comprising the lotus leaf-derived exosomes as described above.

In one embodiment of the present invention, the anti-inflammatory composition further comprises a vehicle.

In another aspect, the present invention also provides a use of the lotus leaf-derived exosomes in preparing an anti-inflammatory or wound-healing drug, composition, or nutritional supplement.

The present invention has the following advantages: the present invention discloses extracting lotus leaf-derived exosomes by different separation methods and verifies the existence of lotus leaf-derived exosomes. In addition, the features of the lotus leaf-derived exosomes obtained were identified, and purification methods and qualitative standards were established for lotus leaf-derived exosomes. The lotus leaf-derived exosomes obtained by the foregoing methods have not only similar particle sizes, but also zeta potentials that indicate stability. It has also been proved by experimentation that lotus leaf-derived exosomes can be used to alleviate inflammatory response or heal wounds and can be further used to prepare anti-inflammatory or wound-healing drugs, compositions, or nutritional supplements.

### BRIEF DESCRIPTION OF THE SEVERAL VIEWS OF THE DRAWINGS

The aforesaid and other objectives, features, and advantages of the present invention can be better understood by referring to the following detailed description of some embodiments of the invention in conjunction with the accompanying drawings, in which:

FIG. 1 shows the process flow of a sample pretreatment intended for the lotus leaf-derived exosomes of the invention;

FIG. 2 shows the particle size distributions of lotus leaf-derived exosomes extracted with different methods, with FIG. 2, A showing the distribution corresponding to a polymer precipitation method, FIG. 2, B showing the distribution corresponding to an ultrafiltration method, FIG. 2, C showing the distribution corresponding to a size-exclusion chromatographic method, FIG. 2, D showing the distribution corresponding to an ultracentrifugation method, FIG. 2, E showing the distribution corresponding to a density-gradient centrifugation method and to the 30-45% sucrose solution layer, and FIG. 2, F showing the distribution corresponding to the density-gradient centrifugation method and to the 45-60% sucrose solution layer;

FIG. 3 shows the observation results of the appearances of lotus leaf-derived exosomes extracted with different methods, with FIG. 3, A showing the result corresponding to a polymer precipitation method, FIG. 3, B showing the result corresponding to an ultrafiltration method, FIG. 3, C showing the result corresponding to a size-exclusion chromatographic method, FIG. 3, D showing the result corresponding to an ultracentrifugation method, FIG. 3, E showing the result corresponding to a density-gradient centrifugation method and to the 30-45% sucrose solution layer, and FIG. 3, F showing the result corresponding to the density-gradient centrifugation method and to the 45-60% sucrose solution layer;

FIG. 4 is a graph showing the zeta potentials of lotus leaf-derived exosomes extracted with different methods;

FIG. 5 shows the experimental results of samples taken from lotus leaf-derived exosomes extracted with a combined method, with FIG. 5, A showing the result of the final density-gradient centrifugation, FIG. 5, B showing particle size distributions corresponding to the combined method, FIG. 5, C showing the yields of the lotus leaf-derived exosomes produced by the combined method, and FIG. 5, D showing zeta potentials corresponding to the combined method;

FIG. 6 shows the experimental results of the anti-inflammatory effects of lotus leaf-derived exosomes extracted with different methods, with FIG. 6, A showing the cell viability of RAW264.7 macrophages after the intervention of a lipopolysaccharide (LPS), and FIG. 6, B showing the result of a nitrite test on the anti-inflammatory effects of the intervention of lotus leaf-derived exosomes in RAW264.7 macrophages having an LPS-induced inflammatory response, wherein the statistical analysis was carried out through one-way analysis of variance (one-way ANOVA) and Tukey's post-hoc analysis, and the TFF, UC, and SEC-group data show significant differences (p<0.05) from the LPS-group data; and

FIG. 7 shows the results of an in vitro test on the wound-healing ability of the lotus leaf-derived exosomes of the invention, with FIG. 7, A showing time-lapse microscopic images showing the migration distances of HaCaT cells given the intervention of lotus leaf-derived extracellular vesicles (LDEVs) at different concentrations, and FIG. 7, B showing changes in wound area, wherein the statistical analysis was carried out through one-way ANOVA and Tukey's post-hoc analysis, and the LDEV-group data show significant differences (p<0.05) from the control-group (PBS-group) data.

### DETAILED DESCRIPTION OF THE INVENTION

The technical contents of the present invention are detailed below with reference to the accompanying drawings. It should be pointed out that, to facilitate description, the drawings are not necessarily drawn to scale, and that the drawings and the proportions shown therein are not intended to limit the scope of the invention.

Unless otherwise defined, all the technical and scientific terms used herein have the same meanings as would generally be understood by a person of ordinary skill in the art. Throughout this application, the following terms have their respective meanings defined as follows:

Unless otherwise specified, the term “or” means “and/or.” The term “include” does not exclude the existence, or addition, of one or more components, steps, operations, or elements apart from, or to, the stated components, steps, operations, or elements. The terms “comprise,” “include,” “contain,” “encompass,” and “have” are interchangeable and non-restrictive. Moreover, as used in this specification and the appended claims, each of the articles “a/an” and “the” includes plural references unless otherwise specified in the context. For example, the terms “a/an,” “the,” “one or more,” and “at least one” are interchangeable herein.

As used herein, the term “lotus leaf” refers to a leaf of a lotus plant.

As used herein, the terms “lotus leaf-derived exosome,” “lotus leaf-derived extracellular vesicle (LDEV),” “lotus leaf-derived exosome-like nanoparticle,” and “lotus leaf-derived exosome nanoparticle” are interchangeable and refer to plant-derived exosome-like nanoparticles extracted from lotus leaves.

One aspect of the present invention provides lotus leaf-derived exosomes that are extracted from lotus leaves by an extraction method selected from the group consisting of: a polymer precipitation method, an ultracentrifugation method, an ultrafiltration method, a density-gradient centrifugation method, and a size-exclusion chromatographic method. In a preferred embodiment, the method by which lotus leaf-derived exosomes are extracted is an ultrafiltration method.

Due to a lack of previous studies on lotus leaf-derived exosomes, the inventor of the present invention attempted to use different methods, or a combination thereof, to extract and purify lotus leaf-derived exosomes, before comparing, and thereby finding the differences between, the features of the lotus leaf-derived exosomes extracted with the different methods. It was found through experiments that the lotus leaf-derived exosomes obtained by the different extraction methods did not differ greatly in particle size. In one embodiment of the invention, the lotus leaf-derived exosomes have particle sizes ranging from 50 to 300 nm, such as but not limited to 50 nm, 63 nm, 70 nm, 84 nm, 90 nm, 100 nm, 115 nm, 126 nm, 132 nm, 136 nm, 152 nm, 159 nm, 163 nm, 166 nm, 200 nm, 217 nm, 223 nm, 235 nm, 248 nm, 251 nm, 266 nm, 273 nm, 286 nm, 298 nm, or 300 nm.

After determining the particle sizes of the lotus leaf-derived exosomes extracted with the different methods, the inventor of the present invention further analyzed the zeta potentials of the lotus leaf-derived exosomes in order to determine the stability of those particles. Generally, as far as colloid particles are concerned, the greater the magnitude of the zeta potential, the greater the thickness of the electric double layer (i.e., the distance from the slipping plane corresponding to a particle to the surface of the particle), and the greater the thickness of the electric double layer, the less likely it will be for adjacent particles to be attracted to each other and coagulate, i.e., the more likely it will be for the colloid particles to stay stably suspended in a colloid solution. Conversely, in a solution of particles with a zeta potential of a small magnitude, the weak repelling forces of the electric charges on the particles tend to result in coagulation (i.e., the formation of large granules) and hence precipitation. Currently, it is generally believed that particles tend to coagulate when their zeta potential is 0±5 mV, that particles are slightly stable when their zeta potential is ±10-30 mV, that particles have moderate stability when their zeta potential is ±30-40 mV, that particles have good stability when their zeta potential is ±40-60 mV, and that particles have excellent stability when their zeta potential is higher than 60 mV or lower than −60 mV. In one embodiment of the invention, the lotus leaf-derived exosomes have a zeta potential lower than −20 mV, such as but not limited to −20 mV, −21 mV, −22 mV, −23 mV, −24 mV, −25 mV, −26 mV, −27 mV, −28 mV, −29 mV, −30 mV, −31 mV, −32 mV, −33 mV, −34 mV, −35 mV, −36 mV, −37 mV, −38 mV, −39 mV, −40 mV, −41 mV, −42 mV, −43 mV, −44 mV, −45 mV, −46 mV, −47 mV, −48 mV, −49 mV, −50 mV, −51 mV, −52 mV, −53 mV, −54 mV, −55 mV, −56 mV, −57 mV, −58 mV, −59 mV, or −60 mV. In a preferred embodiment, the lotus leaf-derived exosomes have a zeta potential lower than −30 mV.

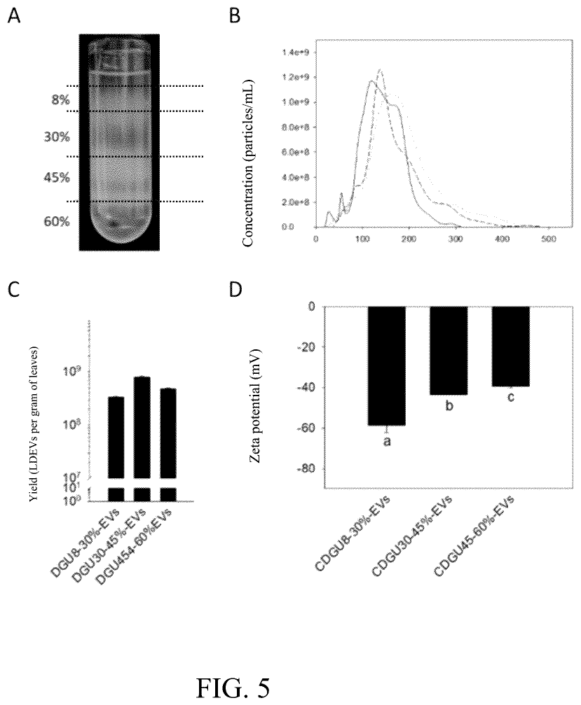

In addition, the inventor of the present invention used a combination of two or more of the aforementioned extraction and purification methods to extract EVs. The results show that the lotus leaf-derived exosomes extracted with a combined method had similar particle sizes to those extracted with a single method, had zeta potentials indicating good stability, and had higher yields than those extracted with a single method (see FIG. 5). Thus, the present invention has established methods for extracting lotus leaf-derived exosomes and identified the different features of the lotus leaf-derived exosomes extracted with the different methods.

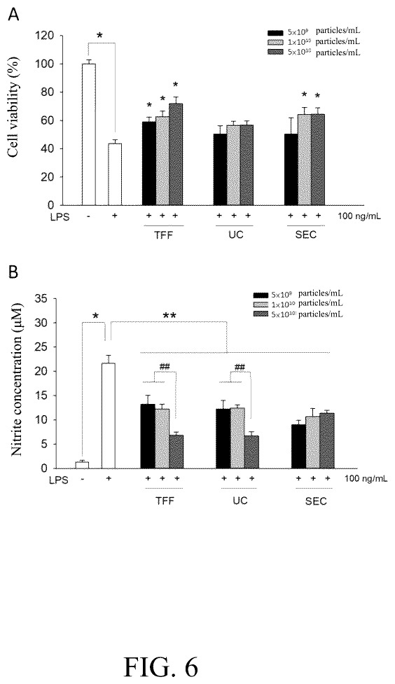

In one embodiment of the present invention, the lotus leaf-derived exosomes are used to alleviate inflammatory response or to heal wounds. In a preferred embodiment, the inflammatory response is caused by a lipopolysaccharide (LPS). As lotus leaves are known to have multiple bioactive substances with antioxidant and anti-inflammatory effects, the inventor of the invention conducted an anti-inflammation experiment in order to find out whether or not lotus leaf-derived exosomes also have an anti-inflammatory effect. The anti-inflammation experiment used an LPS to induce an inflammation mode of RAW264.7 mouse macrophages, and lotus leaf-derived exosomes were given to the macrophages in order to test the anti-inflammatory ability of the exosomes. The test results show that all the lotus leaf-derived exosomes extracted with the different methods were anti-inflammatory and were able to alleviate the LPS-induced inflammatory response (see FIG. 6).

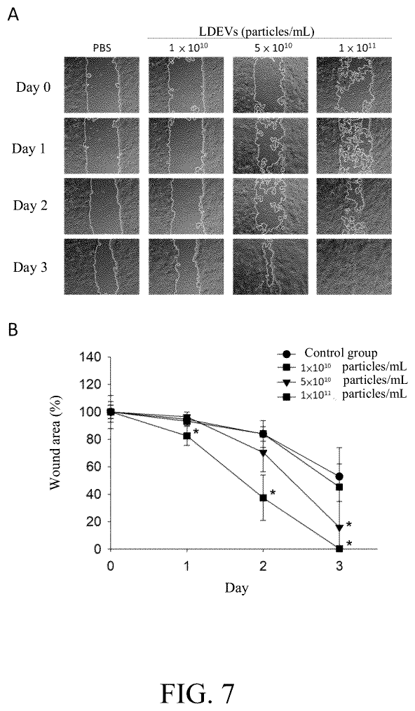

Besides, wound healing is a complicated physiological process that includes a series of inflammatory responses, cell regeneration, cell migration, and so on. The healing process of a skin wound generally starts with an inflammatory response that takes place immediately after the skin is injured. While inflammation helps remove the bacteria and injured tissues around the wound, an excessive inflammatory response may have negative impact on wound healing and delay the repairing process. Therefore, an appropriate intervention of anti-inflammatory treatment to reduce such an excessive inflammatory response can speed up wound healing in addition to mitigating the pain and discomfort caused by inflammation. Now that the inventor of the present invention has found that lotus leaf-derived exosomes have an anti-inflammatory effect, the wound-healing effect of lotus leaf-derived exosomes was further tested. The test results show that the lotus leaf-derived exosomes were indeed effective in healing wounds, and that the higher the concentration of the lotus leaf-derived exosomes, the stronger the wound-healing ability (see FIG. 7). It can therefore be inferred that lotus leaf-derived exosomes have the ability to enhance wound repair and can shorten the time required for wound healing.

In the foregoing experiments, fresh lotus leaves were used as the raw material on which extraction was performed. The present invention, however, is not limited to the use of fresh lotus leaves. Withered or dried lotus leaves may also be used as the raw material on which extraction is performed.

Another aspect of the present invention provides an anti-inflammatory composition that includes the aforesaid lotus leaf-derived exosomes. In one embodiment of the invention, the anti-inflammatory composition further includes a vehicle. That is to say, the composition is mixed with a pharmaceutically acceptable vehicle according to a conventional drug combination technique in order to meet the requirements of a drug preparation process, to facilitate the preparation of a dose of a drug, or to satisfy the requirements of a dosage form. Appropriate pharmaceutically acceptable vehicles are well known to a person of ordinary skill in the art. In addition, the anti-inflammatory composition may include a pharmaceutically acceptable excipient, buffer, or stabilizer, or other ingredients that are well known in the art. These well-known ingredients should be non-toxic and should not interfere with the performance of the active ingredients. The excipient may include one or more surfactants, organic or inorganic salts, diluents, solutizers, thickeners, reducing agents, antioxidants, chelating agents, preservatives, and so on.

In one embodiment of the present invention, the anti-inflammatory composition is applied to vertebrates. In a preferred embodiment, the anti-inflammatory composition is applied to mice. In a more preferred embodiment, the anti-inflammatory composition is applied to humans. The anti-inflammatory composition of the invention can be administered orally or by way of injection, e.g., through intravenous therapy or subcutaneous injection, as a drug for alleviating inflammatory response.

Yet another aspect of the present invention provides a use of lotus leaf-derived exosomes in preparing an anti-inflammatory or wound-healing drug, composition, or nutritional supplement.

### EMBODIMENTS

It should be understood that the examples and embodiments described herein serve only to expound the present invention and to suggest to those skilled in the art various modifications or changes that are within the scope, and do not depart from the spirit, of this application and the appended claims. All the published patent applications, granted patents, and patent applications cited herein, if any, are incorporated herein by reference in their entirety for all purposes.

To find out whether or not lotus leaves contain exosome-like nanoparticles and whether or not these nanoparticles can be successfully separated, the inventor of the present invention used a polymer precipitation method, an ultracentrifugation method, an ultrafiltration method, a density-gradient centrifugation method, a size-exclusion chromatographic method, and a combined method to extract and purify lotus leaf-derived exosome-like nanoparticles, analyzed the particle sizes and concentrations of the nanoparticles with a nanoparticle tracking analyzer, and then observed the appearances of the nanoparticles under an electron microscope. Moreover, once the exosome nanoparticles were separated, a comparison was made to identify the differences among the lotus leaf-derived exosome nanoparticles extracted with the different methods; more specifically, the particle sizes, yields, and appearances of the exosome-like nanoparticles were analyzed.

### Experimental Methods

### 1. Extraction and Purification of Lotus Leaf-Derived Exosome-Like Nanoparticles

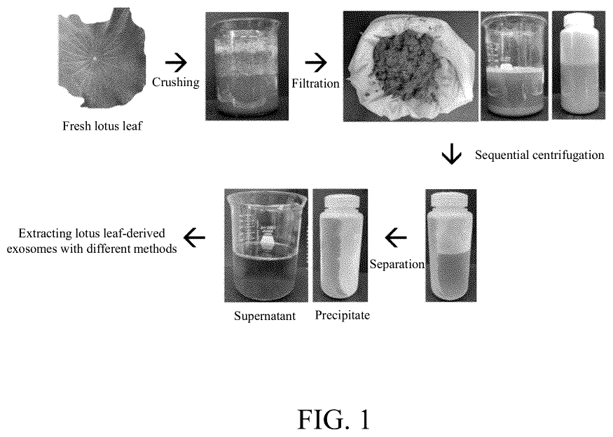

The lotus leaves used in the experiments were samples provided by BO HUI BIOTECH CO., LTD. To successfully extract and separate lotus leaf-derived exosome-like nanoparticles, the lotus leaf samples received a pretreatment (as shown in FIG. 1) prior to the extraction of lotus leaf-derived exosome-like nanoparticles with the different separation methods.

### 1-1. Polymer Precipitation (PEG)

Lotus leaves were added with an appropriate amount of double-distilled water, crushed in a juicer, and then strained through a screen filter having a pore diameter of 300 μm. Following that, the filtrate went through a sequential centrifugation process (2,000×g for 10 minutes; 6,000×g for 30 minutes; and 15,000×g for 60 minutes) to remove large pieces/sections of lotus-leaf tissues and cell fragments, and a polyvinylidene difluoride (PVDF) membrane having a pore diameter of 0.8 μm was subsequently used for filtration (see FIG. 1), before the pH value of the filtrate was adjusted to 5.0 with hydrogen chloride. After that, PEG6000 (with a final concentration of 10%) was added, and the mixed solution was allowed to rest overnight at 4° C. and then centrifuged at 8,000×g for 30 minutes. The precipitate obtained by centrifugation was exosome-like nanoparticles. The exosome-like nanoparticles were suspended in an appropriate amount of double-distilled water, then dialyzed for 24 hours through a 10 kDa dialysis membrane, and then filtered through a membrane filter having a pore diameter of 0.45 μm such that clean exosomes were obtained.

### 1-2. Ultracentrifugation (UV)

Large pieces/sections of lotus-leaf tissues and cell fragments were removed by the same steps as the initial ones of the polymer precipitation method. A PVDF membrane having a pore diameter of 0.8 μm was subsequently used for filtration (see FIG. 1), and the filtrate was centrifuged at 120,000 ×g for 2 hours such that exosome-like nanoparticles were obtained. The exosome-like nanoparticles were washed twice with double-distilled water (centrifuged at 120,000×g for 2 hours), then suspended in an appropriate amount of double-distilled water, and then filtered through a membrane filter having a pore diameter of 0.45 μm such that clean exosomes were obtained.

### 1-3. Ultrafiltration (UF)

Ultrafiltration was performed using the tangential flow filtration (TFF) method. Large pieces/sections of lotus-leaf tissues and cell fragments were removed by the same steps as the initial ones of the polymer precipitation method. A PVDF membrane having a pore diameter of 0.8 μm was subsequently used for filtration (see FIG. 1), and the filtrate was introduced into a TFF system and filtered through hollow-fiber membranes. To start with, filtration was carried out using a hollow-fiber membrane having a pore diameter of 0.65 μm (D02-E65U-07-S). The filtrate was collected and then concentrated with a 750 kDa hollow-fiber membrane. Lastly, solution displacement was performed with double-distilled water (the displacement volume being 1 liter), and clean exosomes were obtained after filtration through a membrane filter having a pore diameter of 0.45 μm.

### 1-4. Density-Gradient Centrifugation (DG)

Exosome-like nanoparticles were obtained by the ultracentrifugation method, and the solution obtained was put on top of a sucrose solution having a concentration gradient (composed of 8%, 30%, 45%, and 60%). After centrifugation at 120,000×g for 2 hours, the solution in the band between the 8% and 30% layers and the solution in the band between the 30% and 45% layers were drawn out and washed twice with double-distilled water (centrifuged at 120,000×g for 2 hours). The exosome-like nanoparticles obtained were then suspended in an appropriate amount of double-distilled water, and clean exosomes were obtained after filtration through a membrane filter having a pore diameter of 0.45 μm.

### 1-5. Size-Exclusion Chromatography (SEC)

Exosome-like nanoparticles were obtained by the ultracentrifugation method and were reconstituted in an appropriate amount of double-distilled water. The resulting suspension was put into a qEV column from above, and separation took place without pressure application. With each mL defining a unit fraction, the solutions in different fractions were collected. After that, the solutions in the fractions where the sample was present were mixed and filtered through a membrane filter having a pore diameter of 0.45 μm for subsequent analysis.

### 1-6. Combined Method

In exosome purification, purified exosomes tend to be contaminated by lipoproteins due to the similarity in size and density between exosomes and lipoproteins. Considering that density-gradient centrifugation is based on differences in density, and that size-exclusion chromatography performs exosome separation based on molecular weights, the inventor of the present invention further evaluated whether or not higher-purity exosomes can be separated by a combination of different methods.

The combined method was essentially a combination of ultrafiltration, density-gradient centrifugation, and size-exclusion chromatography. To begin with, lotus leaf-derived exosome-like nanoparticles were obtained by ultrafiltration. Next, the precipitate obtained by centrifugation was reconstituted in an appropriate amount of double-distilled water, before separation took place in a qEV column without pressure application. The solutions in the fourth to the sixth fractions were collected and mixed, and the mixed solution was put on top of a sucrose solution having a concentration gradient (composed of 8%, 30%, 45%, and 60%). After centrifugation at 100,000×g for 2 hours, the solutions in the band between the 8% and 30% layers, in the band between the 30% and 45% layers, and in the band between the 45% and 60% layers were drawn out, washed twice with double-distilled water (centrifuged at 100,000×g for 2 hours), and then filtered through a membrane filter having a pore diameter of 0.45 μm for subsequent analysis.

### 2. Feature Analysis and Morphological Observation of Lotus Leaf-Derived Exosome-Like Nanoparticles

The analysis of the concentrations and particle size distributions of the lotus leaf-derived exosome-like nanoparticles obtained was entrusted to the Department of Medical Research, National Taiwan University Hospital, and was conducted with a nanoparticle tracking analyzer (NTA) (NanoSight NS300). The zeta potentials of the lotus leaf-derived exosome-like nanoparticles were measured with an instrument for dynamic light-scattering particle size analysis and zeta potential analysis (Zetasizer Nano, Malvern). The morphologies of the purified exosome nanoparticles were observed by transmission electron microscopy (TEM).

### 3. Cell Cultivation and Inflammation Mode Induction

A cell inflammation mode was induced in RAW264.7 mouse macrophages mainly by way of a lipopolysaccharide (LPS). More specifically, RAW264.7 macrophages were seeded in a 24-well plate at a concentration of 5×105 cells per well and were cultured at 37° C. for 12 hours. After that, the Dulbecco's modified Eagle medium (DMEM) used, which contained phenol red, was removed, the cells were washed twice with phosphate-buffered saline (PBS), and then a DMEM without phenol red was added along with the LPS such that the final concentration of the LPS was 100 ng/mL. 10 minutes after the addition of the LPS, lotus leaf-derived exosome-like nanoparticle samples of different concentrations intervened, and the cells were cultured for another 24 hours to complete the inflammation induction test.

### 4. Cell Viability Assay

Cell viability was analyzed through an MTT assay. 24 hours after the inflammation induction test, the existing cell culture medium was removed, and the cells were rinsed twice with PBS and then added with fresh DMEM, plus an MTT reagent at 2 g/L (the final concentration being 10%). After cultivation at 37° C. for 90 minutes, the cell culture medium was removed, and the purple crystals were dissolved with dimethyl sulfoxide (DMSO). The absorbance values at the wavelength of 570 nm were then determined.

### 5. Nitrite Test

When cell inflammation occurred, the affected cells produced NO⁻, which in turn reacted with the culture medium to produce NO₂⁻. Therefore, to assess the degrees of inflammation of the cells cultured, the NO₂⁻ level of the cell culture medium in each well was tested with a Griess reagent. More specifically, after the 24-hour cultivation in the inflammation mode induction test, 100 μL of cell culture medium was collected from each well and then added with the Griess reagent in order for reactions to take place. Once the reactions were completed, the absorbance values at the wavelength of 550 nm were determined. The nitrite concentrations were calculated by substituting each absorbance value obtained from the test into a standard calibration curve prepared with a standard nitrite solution.

### 6. In Vitro Wound Healing Test

The wound healing test was conducted mainly on human keratinocytes (HaCaT cells). First, a two-well culture-insert was placed in each well of a 24-well plate, and each well was seeded with 3×10⁴ cells. The cells were cultured overnight in a high-concentration DMEM containing 10% fetal bovine serum (FBS) in a 37° C. and 5% CO₂ environment. The culture-inserts were removed the next day, and LDEVs of different concentrations were added to the culture medium. Migrations of the HaCaT cells were then observed, photographed, and analyzed. The cells were observed with a cell heating and cultivation system (Ibidi Stage Top Incubator), and the wound areas, which changed with cell migration, were analyzed with ImageJ.

### Experimental Results

### 1. Effects of Different Extraction Methods on the Particle Sizes of Lotus Leaf-Derived Exosomes

To know the effects of the different extraction methods on the separation of lotus leaf-derived exosome-like nanoparticles, the first step was to extract lotus leaf-derived exosomes by the different separation methods, namely polymer precipitation, ultracentrifugation, ultrafiltration, density-gradient centrifugation, and size-exclusion chromatography. The material on which extraction was performed was fresh lotus leaves, and before the extraction began, fresh lotus leaf samples received the pretreatment shown in FIG. 1.

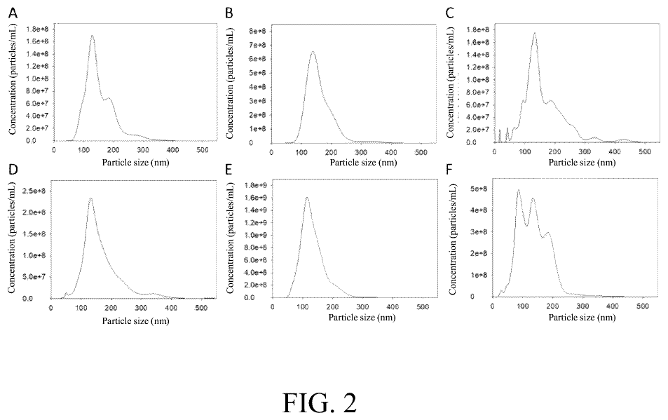

After the sample pretreatment, the different methods were separately used to separate lotus leaf-derived exosomes. FIG. 2 shows the particle size distributions of the lotus leaf-derived exosomes extracted with the different methods. As shown by the results in FIG. 2, the lotus leaf-derived exosome particles obtained by the different extraction methods had similar particle sizes that did not differ greatly from one extraction method to another. The particle sizes were distributed mainly between 50 and 300 nm. The average particle sizes of the lotus leaf-derived exosomes obtained by the different extraction methods are 152.4±49 nm (the EVs extracted by the polymer precipitation method, hereinafter referred to as PEG-EVs for short; see FIG. 2, A), 159.5±48.5 nm (the EVs extracted by the tangential flow filtration method, hereinafter referred to as TFF-EVs for short; see FIG. 2, B), 163.2±70.1 nm (the EVs extracted by the size-exclusion chromatographic method, hereinafter referred to as SEC-EVs for short; see FIG. 2, C), 166.5±64.3 nm (the EVs extracted by the ultracentrifugation method, hereinafter referred to as UC-EVs for short; see FIG. 2, D), about 132.6±39.9 nm (the EV particles obtained from the 30-45% sucrose solution layer of the density-gradient centrifugation method, hereinafter referred to as DGU30-45%-EVs for short; see FIG. 2, E), and about 136.3±52 nm (the EV particles obtained from the 45-60% sucrose solution layer of the density-gradient centrifugation method, hereinafter referred to as DGU45-60%-EVs for short; see FIG. 2, F). Of all the lotus leaf-derived exosomes obtained, DGU30-45% and DGU45-60% had the smallest average particle sizes.

### 2. Effects of Different Extraction Methods on the Physical Properties of Lotus Leaf-Derived Exosomes

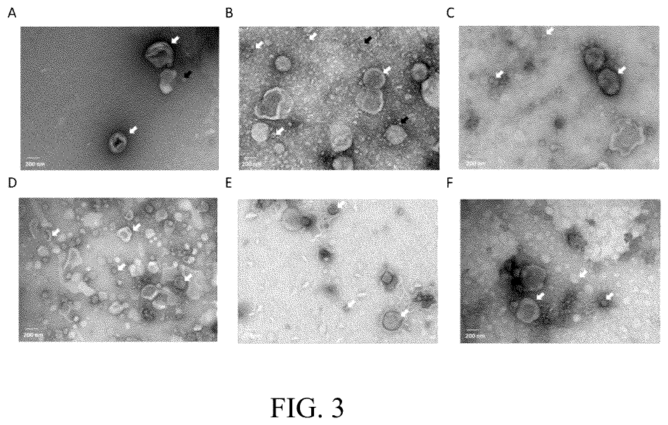

In addition to the particle size analysis by the NTA, the appearances of the EVs obtained were observed under a transmission electron microscope, and particle stability was evaluated with an analysis of zeta potentials. FIG. 3 shows the observation results of the appearances of the lotus leaf-derived exosomes extracted with the different methods. It can be seen in FIG. 3, A to FIG. 3, F that there are noticeable cup-like particle structures (indicated by the white arrows in the images), which are typical structures to be found when negatively stained EVs are observed under an electron microscope. Such special structures were formed while the samples were being dried, during which process the surface of a vesicle might sank toward the center of the vesicle because of dryness. The TEM observation results are similar to the NTA analysis results in terms of particle size and particle size distribution, and this proves that the particle sizes obtained by the NTA are consistent with the actual particle sizes, and that the particles were EVs.

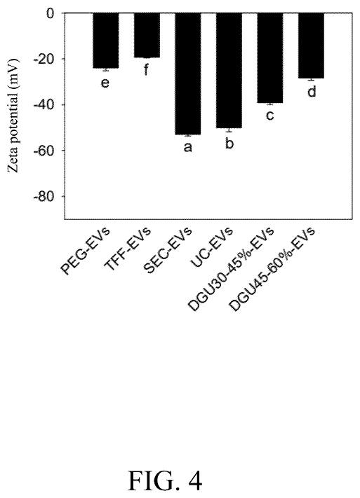

In the analysis of the stability of the lotus leaf-derived exosomes obtained, the zeta potential was used to determine the features and stability of the exosomes, and the results are shown in FIG. 4. The zeta potentials of all the lotus leaf-derived exosomes extracted with the different methods were lower than −20 mV, indicating that the lotus leaf-derived exosomes extracted with the different methods were relatively stable. In particular, the zeta potentials of SEC-EVs, UC-EVs, DGU30-45%-EVs, and DGU45-60%-EVs were lower than −30 mV.

### 3. Effects of Different Extraction Methods on the Yield of Lotus Leaf-Derived Exosomes

To know the effects of the different extraction methods on the yield of lotus leaf-derived exosomes, the yields of the lotus leaf-derived exosomes obtained were further analyzed. Referring to Table 1, which shows the analysis result of the yields of the lotus leaf-derived exosomes extracted with the different methods, the tangential flow filtration method produced the highest yield (about 3.69±0.02×10⁹) of lotus leaf-derived exosomes, followed by the ultracentrifugation method and the PEG precipitation method, the yields of which two methods are 2.20±0.13×10⁸ and 2.13±0.07×10⁸ respectively. The size-exclusion chromatographic method produced the lowest yield (about 4.62±0.06×10⁷) of lotus leaf-derived exosomes. In addition, a comparison of the yields of DGU30-45%-EVs and DGU45-60%-EVs reveals that the yield of DGU30-45%-EVs is approximately two times that of DGU45-60%-EVs. As the density-gradient centrifugation method is equivalent to separating total EVs even further, it can be known from the density-gradient centrifugation result that the lotus leaf-derived exosome particles extracted according to the present invention had densities mostly in the range from 1.13 to 1.2 g/mL. Since this range is considered to be the density range of the exosomes of animal cells, it can be inferred that the LDEV particles extracted according to the invention are exosome-like nanoparticles.

TABLE 1
Yields of the lotus leaf-derived exosomes extracted
with different extraction methods

| | Yield |
|---|---|
| | (meantstandard deviation; |
| | number of particles per gram |
| Method | of fresh leaves |
| PEG | 2.13 ± 0.07 × 10⁸, b |
| TFF | 3.69 ± 0.02 × 10⁹, a |
| SEC | 4.62 ± 0.06 × 10⁷, d |
| UC | 2.20 ± 0.13 × 10⁸, b |
| DGU30-45% | 1.14 ± 0.001 × 10⁸, c |
| DGU45-60% | 5.22 ± 0.48 × 10⁷, d |

### 4. Effects of Extracting and Purifying Lotus Leaf-Derived Exosomes With the Combined Method

Literature has shown that using a combination of two or more extraction and purification methods to extract and purify EVs is advantageous to enhancing the purity of the EV sample obtained. Therefore, to increase the yield of the lotus leaf-derived exosomes of the present invention, the inventor of the invention further used a combination of ultrafiltration, density-gradient centrifugation, and size-exclusion chromatography to separate and purify lotus leaf-derived exosomes. The purified samples and their particle size distributions are shown in FIG. 5. As the combined method includes density-gradient centrifugation, there were a plurality of final samples. The samples taken from different sucrose concentration layers were all analyzed. FIG. 5, A shows the result of the final density-gradient centrifugation, and it can be seen in FIG. 5, A that after the centrifugation, noticeable bands appeared in three fractions, namely the fraction between the 8% and 30% layers (hereinafter referred to as the 8-30% fraction for short), the fraction between the 30% and 45% layers (hereinafter referred to as the 30-45% fraction for short), and the fraction between the 45% and 60% layers (hereinafter referred to as the 45-60% fraction for short).

The samples collected from these three fractions had their particle sizes measured with the NTA, and according to the measurement results, the lotus leaf-derived exosomes corresponding to the 30-45% fraction had the largest average particle size while the LDEVs corresponding to the 8-30% fraction had the smallest average particle size. More specifically, the average particle sizes of the EVs obtained by combined-method density-gradient centrifugation (CDGU) and corresponding to the 8-30%, 30-45%, and 45-60% fractions (abbreviated as CDGU8-30%-EVs, CDGU30-45%-EVs, and CDGU45-60%-EVs respectively) are 141.8±44.3 nm, 189.6±75.4 nm, and 170±64.6 nm respectively (see FIG. 5, B). When it comes to yield, CDGU30-45%-EVs had the highest yield (7.93±0.46×10⁸ particles per gram of fresh leaves), followed by CDGU45-60%-EVs (about 4.87±0.19×10⁸ particles per gram of fresh leaves), and CDGU8-30%-EVs had the lowest yield (about 3.38±0.16×10⁸ particles per gram of fresh leaves) (see FIG. 5, C). A comparison of zeta potentials further shows that CDGU8-30%-EVs had the greatest potential magnitude (−60 mV), that the potentials of the other two samples were about −40 mV, and that in consequence all the samples had good particle stability (see FIG. 5, D).

### 5. Alleviation of LPS-Induced Inflammation of Mouse Macrophages by LDEVs

To know whether or not LDEVs have an anti-inflammatory effect, an anti-inflammation test was conducted by inducing an inflammation mode of RAW264.7 mouse macrophages with an LPS, and the experimental results are shown in FIG. 6. When the LPS was added during cell cultivation, the cell viability of the RAW264.7 macrophages was significantly reduced by the intervention of the LPS. However, when the LDEVs obtained by the TFF or SEC purification method were also added, cell viability rose significantly and increased with the concentration of the LDEVs (see FIG. 6, A). Furthermore, the nitrite concentrations in the cells were tested with the Griess reagent, and it was found that the intervention of the LDEVs in the RAW264.7 cells under LPS induction caused a significant reduction of the inflammatory response induced by the LPS, and that the nitrite concentrations were lowered as the concentration of the LDEVs increased (see FIG. 6, B). The experimental results also show that the reduction in nitrite concentration was indeed associated with the intervention of the LDEVs. The LDEVs obtained by the UC method failed to reduce the lowering of cell viability caused by the LPS but still had an anti-inflammatory effect. All in all, the foregoing results demonstrate that regardless of the extraction and purification method used, the resulting LDEVs had an anti-inflammatory effect, with the LDEVs obtained by TFF having the highest anti-inflammatory ability.

### 6. In Vitro Wound-Healing Ability of LDEVs

To know whether or not LDEVs have the ability to repair cells and promote wound healing, an in vitro cell migration test was performed on human keratinocytes (HaCaT cells), and the test results are shown in FIG. 7. The intervention of LDEV samples of different concentrations sped up the reduction in wound area significantly, and the cell migration speed increased with the concentration of the sample (see FIG. 7, A). The wound treated with the concentration of 1×10¹¹ particles/mL healed completely at the end of the third day (see FIG. 7, B) whereas the wound treated with PBS showed only a 40% reduction in area at the end of the third day. It can be inferred from the experimental results that LDEVs have the ability to enhance wound repair and shorten the time required for wound healing.

Thus, it has been proved that lotus leaf-derived exosomes can be extracted by different methods, and that the tangential flow filtration system in the ultrafiltration method is particularly effective in extracting and purifying lotus leaf-derived exosomes, with a yield ten times as high as those of the other methods.

According to the above, the present invention discloses extracting lotus leaf-derived exosomes by different separation methods and verifies the existence of lotus leaf-derived exosomes. In addition, the features of the lotus leaf-derived exosomes obtained were identified, and purification methods and qualitative standards were established for lotus leaf-derived exosomes. The lotus leaf-derived exosomes obtained by the foregoing methods have not only similar particle sizes, but also zeta potentials that indicate stability. It has also been proved by experimentation that lotus leaf-derived exosomes can be used to alleviate inflammatory response or heal wounds and can be further used to prepare anti-inflammatory or wound-healing drugs, compositions, or nutritional supplements.

While the present invention has been detailed above, the embodiments described herein are only some preferred ones of the invention and should not be viewed as restrictive of the scope of the invention. Any equivalent change or modification based on the appended claims shall fall within the scope of the invention.

1. Lotus leaf-derived exosomes, extracted from lotus leaves by an extraction method selected from the group consisting of: polymer precipitation, ultracentrifugation, ultrafiltration, density-gradient centrifugation, and size-exclusion chromatography.

2. The lotus leaf-derived exosomes of claim 1, wherein the extraction method is the ultrafiltration.

3. The lotus leaf-derived exosomes of claim 1, wherein the lotus leaf-derived exosomes have particle sizes in a range from 50 to 300 nm.

4. The lotus leaf-derived exosomes of claim 1, wherein the lotus leaf-derived exosomes have a zeta potential lower than −20 mV.

5. The lotus leaf-derived exosomes of claim 4, wherein the zeta potential of the lotus leaf-derived exosomes is lower than −30 mV.

6. The lotus leaf-derived exosomes of claim 1, wherein the lotus leaf-derived exosomes are used to alleviate inflammatory response or heal a wound.

7. The lotus leaf-derived exosomes of claim 6, wherein the inflammatory response is caused by a lipopolysaccharide (LPS).

8. An anti-inflammatory composition, comprising the lotus leaf-derived exosomes of claim 1.

9. The composition of claim 8, further comprising a vehicle.

10. A Use of the lotus leaf-derived exosomes of claim 1 in preparing an anti-inflammatory or wound-healing drug, composition, or nutritional supplement.
